(19) United States
(12) Patent Application Publication
(10) Pub. No.: US 2012/0058509 A1
(43) Pub. Date: Mar. 8, 2012

(54) APPARATUS AND METHOD FOR AFFIXING FROZEN TISSUE SECTIONS TO GLASS OR MEMBRANE MICROSCOPE SLIDES

(76) Inventors: Eric Jeffords Leininger, New Orleans, LA (US); Warnakulasuriya Akash Fernando, New Orleans, LA (US)

(21) Appl. No.: 12/877,229

(22) Filed: Sep. 8, 2010

(51) Int. Cl. G01N 33/48; C12M 1/00

(52) U.S. Cl. 435/40.52; 435/283.1

(57) ABSTRACT

This invention is an apparatus and method for affixing frozen tissue sections to microscope slides. It has three elements; teasing implements, an affixing block and a heating element. The elements are used inside a cryostat prior to processing for molecular biology procedures such as immunohistochemistry or laser micro dissection. The tissue sections are collected onto an affixing block which has indentations that decrease the contact surface area while providing structural support. The tissue sections are then transferred onto a slide via transient application of a heating element to the back of slide. The invention decreases folding of tissue sections, increases morphological consistency, enables the placement of many sections on one slide and enables the adherence of tissue sections with greater thickness.

### FIELD OF INVENTION

The suggested field of invention for this invention is CHEMISTRY: MOLECULAR BIOLOGY AND MICROBIOLOGY CLASS #435 and the suggested subclasses are APPARATUS #283.1 and subclass IMMUNOHISTOCHEMICAL ASSAY #935.

### BACKGROUND ART

In order to perform some forms of immunohistochemistry and laser micro dissection, there is a relatively common technique for collecting the tissue in molecular biology laboratories. First, a region of interest (ROI) is isolated through resection of animal body part. Second, the ROI is placed inside a frozen tissue embedding medium. This embedding medium usually contains polyethylene glycol and is water-soluble. Third, the embedding medium and ROI themselves are placed within a mold. Finally, after placing the mold in contact with liquid nitrogen, the ROI and medium is rapidly frozen. After being frozen, the ROI/embedding medium complex structurally supports the sample for subsequent sectioning at below freezing temperatures.

In order to slice the frozen ROI into micron-thick sections necessary for immunohistochemistry and laser micro dissection, a cryostat is used. A cryostat is a sectioning machine that is kept below freezing so that the embedding medium remains solid and thin slices of tissue may be gathered from the ROI individually and subsequently affixed to slides. The ROI sections range from 2-500 μM, and are composed of both tissue slices and surrounding embedding medium.

There are two methods used for affixing the tissue sections to the slide inside the cryostat. The first of these is by swooping a room temperature glass or membrane slide upon a ribbon-like collection of tissue sections resting frozen in the cryostat (described herein as the “swooping heat method”). The heat from the slide causes the embedding media and tissue sections to rapidly melt, causing the tissue sections to remain adherent to the slides through electrostatic force. The second of these is to use an adhesive to collect and transfer tissue sections to special adhesive coated glass slides by UV cross-linking (described herein as the “UV method”).

Currently, the “swooping heat method” is the most widely used, and while it is inexpensive relative to the “UV method”, there is a steep learning curve in order to perform this method properly without tissue section folding. The motion a person is required to perform with the “swooping heat method” is a precisely angled swoop of the glass slide to the tissue section ribbon, so that the tissue sections remain flat on the slide as they melt. If the tissue sections are curled, which is dependent on the temperature gradient between the cryostat and the room, or dependent on the incident angle between the slide and ribbon of sections, the tissues will improperly fold onto the slide. This can cause many problems. For example, if the tissue sections are folded, the architecture of the tissue, upon subsequent processing and imaging, is difficult to interpret. Further, folded tissue allows the embedding medium to double up, which can lower the cutting distance of the laser through the tissue section during laser micro dissection. In addition, improper tissue folding will tend to have the sections fall off the slide as the less tissue surface area means less electrostatic interaction between the tissue and the slide upon subsequent processing. Finally, if the angle of the slide used to swoop down and pick up the tissue ribbon is grossly inaccurate or the sections are extensively curled then (for example, when a beginner is attempting to perform the procedure) then the tissue sections can become stacked one on top of the other and further processing would be ill-advised. Overall, in order to perform the “swooping heat method” without improper tissue folding, it takes tens-hundreds of hours of training.

Besides improper tissue folding, another problem with the “swooping heat method” is that, if done improperly, large amounts of tissue sections can be lost. This is because the both the ribbon of tissue sections and glass slide are lengthwise in nature and it is not economically feasible to collect only one section per slide for most experiments. Therefore, if the swooping process does not accurately lay down the sections in the desired manner, swaths of tissue sections are lost, and the user can lose valuable data. Further, though the tissue sections may be small in surface area relative to the slide and multiple samples are being collected on one slide, the procedure still uses many slides as the number of tissue sections that can normally fit on one slide in one horizontal plane is limited. The more slides that are used the more reagents (for example antibodies) are necessary in order to process the tissue further down the line, raising the expense necessary to perform the procedure.

In addition to improper tissue folding and lost sections, the “swooping heat method” may induce micro-folding of tissue structures within an individual section. Thus, although the tissue section appears to lay flat on the slide, individual layers of tissue, for example, bone, epidermis or mesenchyme may themselves be dissociated or overlap one another.

Another problem with the “swooping heat method” is that sections >25 μM (for performing whole-mount immunohistochemistry, or for collecting larger tissue volumes for laser micro dissection) are less able to maintain their adhesion, due to the large amount of embedding medium that interrupts the electrostatic interaction of the tissue and the slide. Subsequent washing and processing of the tissue, removes not only the embedding medium but the thicker tissue sections themselves.

The “swooping heat method” is used not only on glass slides but also on membrane slides. Membrane slides are primarily used for affixing frozen tissue section in order to perform laser micro dissection. The membrane on the membrane slide is a thin film and this is the substrate upon which the tissue sections adhere. This allows laser cutting of both the tissue section and the membrane which can then be selected for ribonucleic acid (RNA) processing. Rather than the room temperature glass slide, a room temperature plastic insert is placed onto the back of the membrane surface opposite the side used for adherence. Similar to the motion of the glass slide, the room temperature membrane slide and plastic insert are swooped down to adhere to a ribbon of tissue causing adherence. Without the plastic insert, the membrane portion of the membrane slide, when inserted into the cryostat, quickly adapts to the inside temperature and the frozen tissue sections will not adhere to the slide.

While the problems inherent in the swooping “heat method” for glass slides are the same for membrane slides, there is an additional complication that arises for membrane slides. This complication arises because the subsequent processing of the tissue sections for laser micro dissection involves the goal of RNA stabilization. At room temperature and in aqueous solutions RNA degrades rapidly due to endogenous RNAses in cells, therefore it is important to perform laser micro dissection on the tissues as rapidly as possible following slide affixing. However, because the water soluble embedding medium, present on the membrane with the tissue section, needs to be removed so that it does not inhibit laser penetration of the tissue, the subsequent processing of the tissue must involve addition of an aqueous solution. It has been shown that RNAse activity is potentiated in aqueous solutions, therefore removal of the embedding medium by washing the tissue in an aqueous solution, enhances degradation of the RNA.

The other primary method of affixing tissue sections to slides for immunohistochemistry, the “UV method”, involves the use of adhesives and a transient pulse of ultraviolet light in order to cross-link the tissue to the slide. In short, an adhesive tape is applied to the section block prior to each individual swipe of the blade and after the blade has passed the section remains affixed to the tape. Subsequently, the tape and individual tissue section themselves are affixed to a special adhesive-coated slide by being mechanically pressed with a rolling device inside the cryostat. This combination, tape/section/slide, are then exposed to transient UV light which cross links the tissue to the slide, so that the tape can be removed and the tissue section remains on the slide. The advantage of this method over the heat swooping method is that there is usually considerably better morphology and no folding.

However, the “UV method” has some problems as well. For example, very few sections can fit on one slide making the collection of tissue slower than the “swooping heat method”. Further , the equipment needed is quite specific and expensive including special tape that can operate at 0° to −40° C., special slides coated with an adhesive necessary to transfer the section, and a low temperature, cryostat-embedded UV light used for cross linking Additionally, these packaged slides are not Rnase free.

### SUMMARY

This invention enables one to transfer frozen tissue sections to a glass or membrane slide in a novel and efficient manner. It is primarily made up of three elements; teasing implements, an affixing block and heating element. After cutting one or more tissue sections, the embedding medium is removed with the teasing implements and transferred to the affixing block. A slide is then placed on top of the affixing block and tissue sections, flattening out the sections. Then a heating element is briefly applied to the back of the slide causing the tissue sections to transfer from the affixing block and adhere to the slide.

The invented apparatus allows tissue sections to remain flat and adherent on the glass or membrane slide with little difficulty. Further, the embedding medium is removed prior to affixing the tissue section to the slide so that micro-folding of tissues within a section does not occur and coagulation of embedding medium does not need to removed prior to laser micro dissection, enabling greater RNA stability. In addition, thick tissue sections (>25-200 μM) easily adhere to the slide. Also, many sections can placed on one slide decreasing necessary reagents and increasing economic efficiency in the laboratory.

### BRIEF DESCRIPTION OF DRAWINGS

FIG. 1. Tissue sections (4) are separated (arrow) from the embedding medium (3; horizontal hatched) on a nominal surface area (5; square hatched) inside the cryostat by means of a teasing implement (1) with a pointed tip (2).

FIG. 2. Tissue sections (4) are placed onto the grooved surface (7) of the affixing block (6) with the teasing implement (1).

FIG. 3. A cross sectional plane through any region of FIG. 2 that contains a tissue section. The cross section shows a representative tissue section (4) lying on top of the grooved surface (7) of the affixing block. The tissue section is folded such that some portions of the section rise above the surface of the affixing block, a common occurrence when collecting tissue. The peaks (8) of the affixing surface (7) contact the tissue section (4) while the groove indentations (9) do not.

FIG. 4. A glass or membrane slide (10) is placed on top of both the tissue sections (4) and the affixing block (6); the square box indicates plane of cross section.

FIG. 5. A cross sectional plane through any region of FIG. 4 that contains a tissue section. The cross sectional plane shows the slide (10) flattening the tissue section (4) on top of the peaks (8) of the grooves of the affixing surface (7) of the affixing block. This flattens the tissue section while minimizing the contact surface area and avoiding contact with the bottom of the grooves (9).

FIG. 6. Heating element (12) is placed on top of the slide (10), the tissue sections and the affixing block (6).

FIG. 7. A cross sectional plane through any region of FIG. 6 that contains a tissue section. The cross sectional plane shows the following structural arrangement: The heating element (12) is on top of the slide (10), which is on top of the tissue sections (4), which are on top of the affixing block (6). Also, in this embodiment, the heat source is room temperature liquid (13; diagonally hatched and shaded) which composes the interior of the heating element (12). This is done for a period of time (1-1000 seconds) in order to make the flattened tissue sections adhere to the slide.

FIG. 8. The heating element (12) is removed (top arrow) and the slide (10) is subsequently removed (bottom arrow) from the affixing block. The tissue sections (4) are now flattened and adherent to the slide (10) and subsequent procedures such as laser micro dissection and immunohistochemistry can now be performed on the sections.

### DETAILED DESCRIPTION OF INVENTION

The function of this apparatus is to affix frozen tissue sections to glass or membrane microscope slides. The apparatus contains several parts; teasing implement(s) for removing the tissue section from the surrounding embedding medium, an affixing block with one or more affixing surfaces which enables the sections to be flattened and transferred to the slide and a heating element which induces transference of the section from the affixing block to the slide.

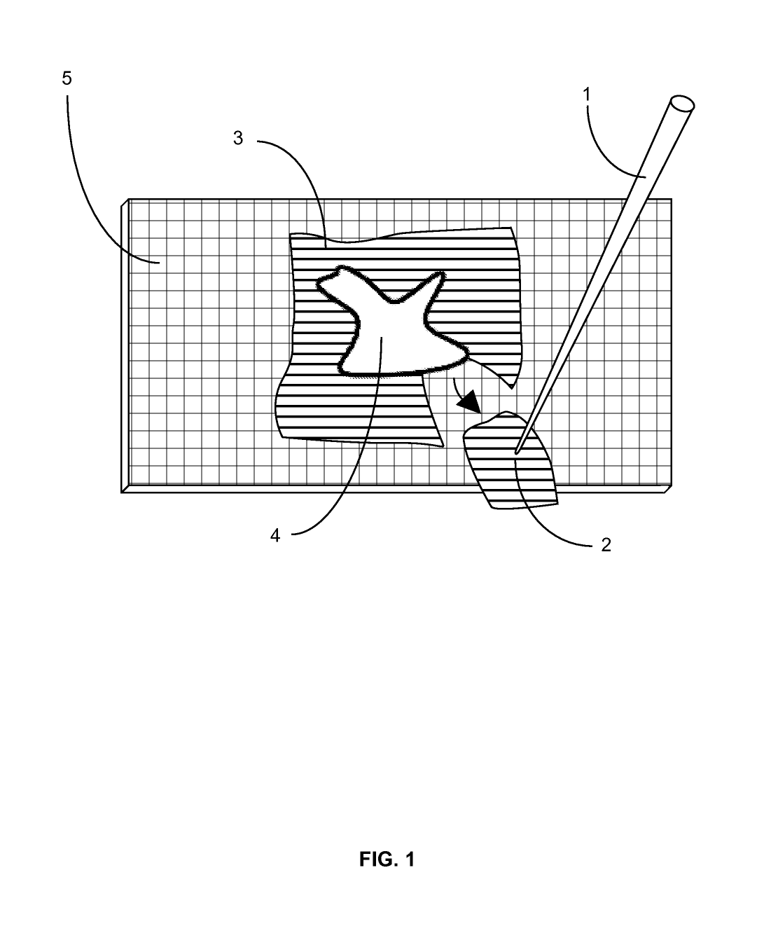

First, after cutting each tissue section, an individual first removes the surrounding embedding medium from the tissue section (FIG. 1). This is done inside the cryostat and with temperature-equilibrated teasing implement(s). The teasing implement (FIG. 1; 1) is small enough to be easily manipulated by hand and has a pointed tip (FIG. 1; 2) so that the surrounding embedding medium (FIG. 1; 3) can be removed from the tissue section (FIG. 1; 4) on a nominal surface (FIG. 1; 5), which provides support, inside the cryostat. This is done visibly by eye or with the aid of an attached magnifying glass. Using the teasing implement and laterally pulling (FIG. 1; arrow) on the medium will separate the medium from the tissue section. After removal of the embedding medium, only the tissue section should remain. The removal of the embedding medium serves three functions. First, when transferring tissue sections from the affixing block to the slide, the heat implement-induced melting of the embedding medium inhibits transference of the tissue section to the slide by filling in the indentations on the surface of the affixing block. Second, if desired (for example to perform laser micro dissection) anhydrous solutions can solely be used to process the tissue, inhibiting RNAases. Third, removal of the surrounding embedding medium frees up space on the slide so that more tissue samples can be afffixed to one slide making down stream applications more economical.

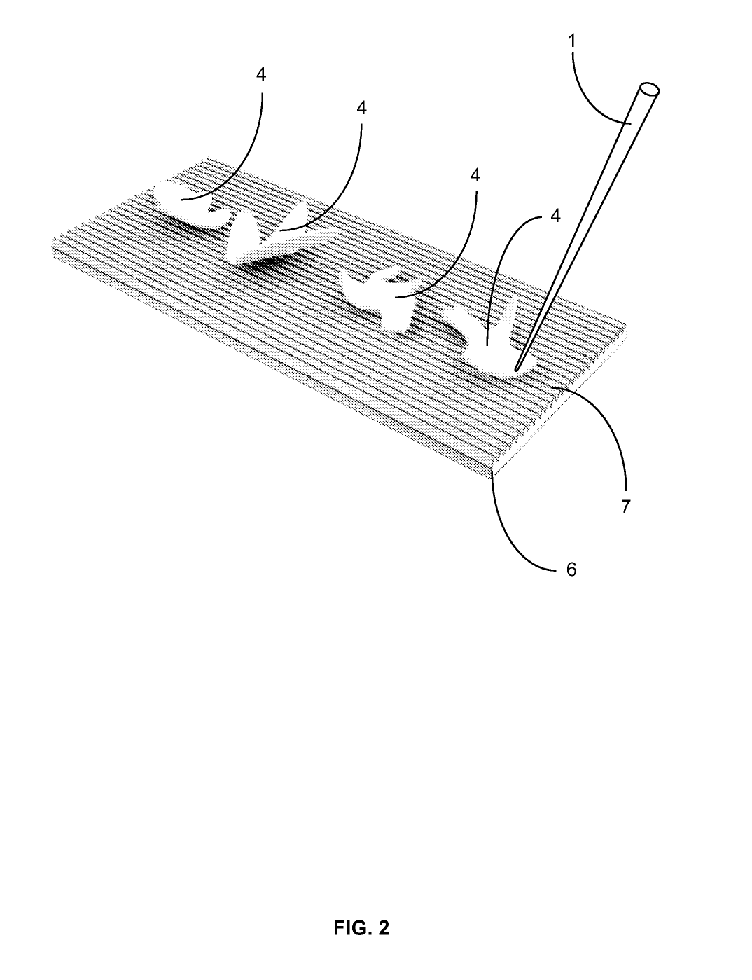

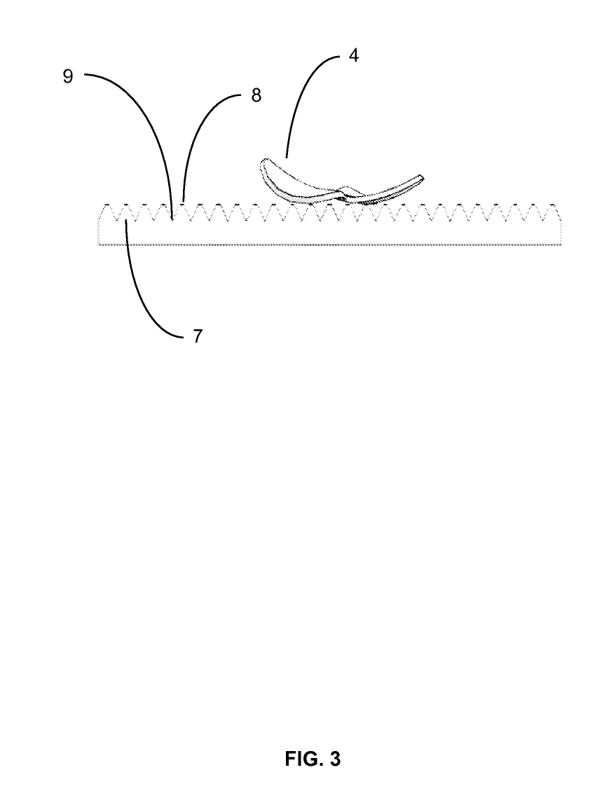

After removal of the embedding medium, the tissue section is transferred to the temperature-equilibrated affixing block (FIG. 2; 6) also inside the cryostat (FIG. 2). The tissue section (FIG. 2; 4)is transferred by a teasing implement (FIG. 2; 1) with its pointed end (FIG. 2; 2), by poking a region of the tissue and causing the tissue to adhere to the teasing implement. The pointed end should be small enough to do no significant damage to the tissue. The tissue section (FIG. 3; 4) will lie on the affixing block (FIG. 3; 6) and the affixing surface (FIG. 3; 7) which in this embodiment has indentations (specifically grooves) (FIG. 3; 9 whose peaks (FIG. 3; 8) support the curled tissue minimizing the contact surface area between the tissue and the affixing surface. Alternatively, one can transfer the tissue section and surrounding medium to the affixing block, by poking the embedding medium, instead of the tissue, prior to separation. Separation of the embedding medium from the tissue section can then be done performed on the affixing block, as long as pieces of embedding medium are not present during heat-induced affixing of the tissue section to the slide. The cutting, separating and transference procedures should be done as many times as is desired for the number of tissue sections per slide.

The affixing block, upon which the tissue sections are placed, should at least be the size of one slide. Further, the affixing block should be covered at least on one slide by a series of indentations, such as grooves. The purpose of the indentations is to decrease the amount of surface area that the affixing block touches the tissue section while still providing structural support for the tissue. In general, the indentations should be small enough to provide structural support and large enough (width and depth-wise) to minimize the contact surface area between the tissue section and affixing block. Minimizing the contact surface are is important for the subsequent process of heat-induced transference of the tissue sections from the affixing block to the slide. If the surface area contact is too great, for instance an affixing block without indentations, than heat-induced transference of the tissue sections from the affixing block to the slide is inhibited.

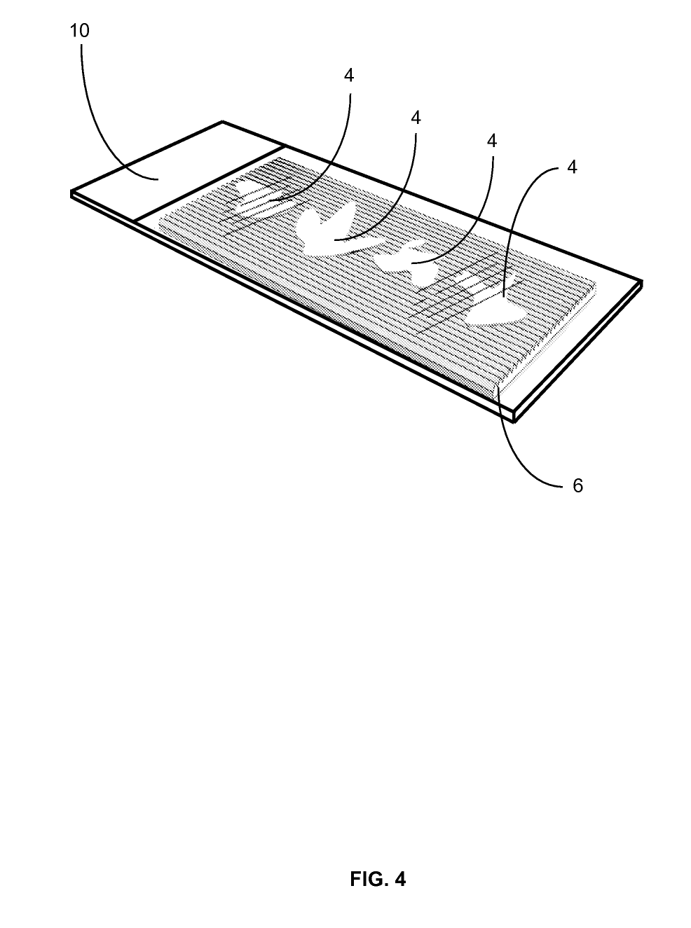

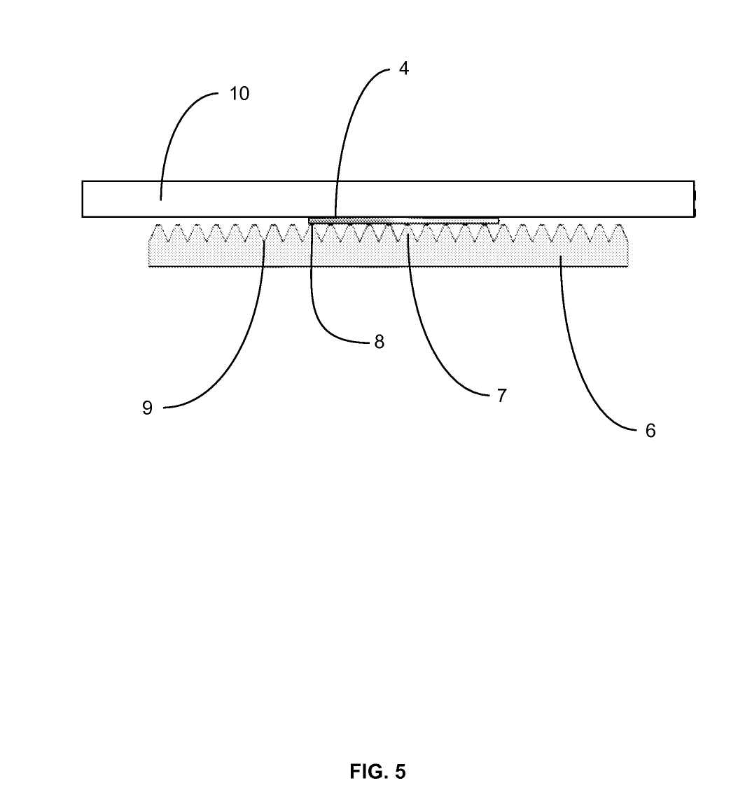

After the desired amount of sections have been placed on the affixing block, a temperature equilibrated slide (FIG. 4; 10) is placed on top of the tissue sections (FIG. 4; 4) and affixing block (FIG. 4; 6). The side of the slide that is placed in contact with the affixing block, should be the front side (usually labeled with glass slides, or indicated as such with membrane slides). The placing of the slide (FIG. 5; 10) on top of the affixing block (FIG. 5; 6) and tissue sections (FIG. 5; 4) will flatten the tissue sections, but the indentations (FIG. 4; 9) on the affixing surface (FIG. 5; 7) will minimize the contact surface area between the affixing surface and the tissue section. This is an advantage over the aforementioned heat swooping methods, in that the weight of the slide will flatten the section so that upon subsequent adherence, folding of the tissue sections is greatly induced and morphological structure is maintained within the section.

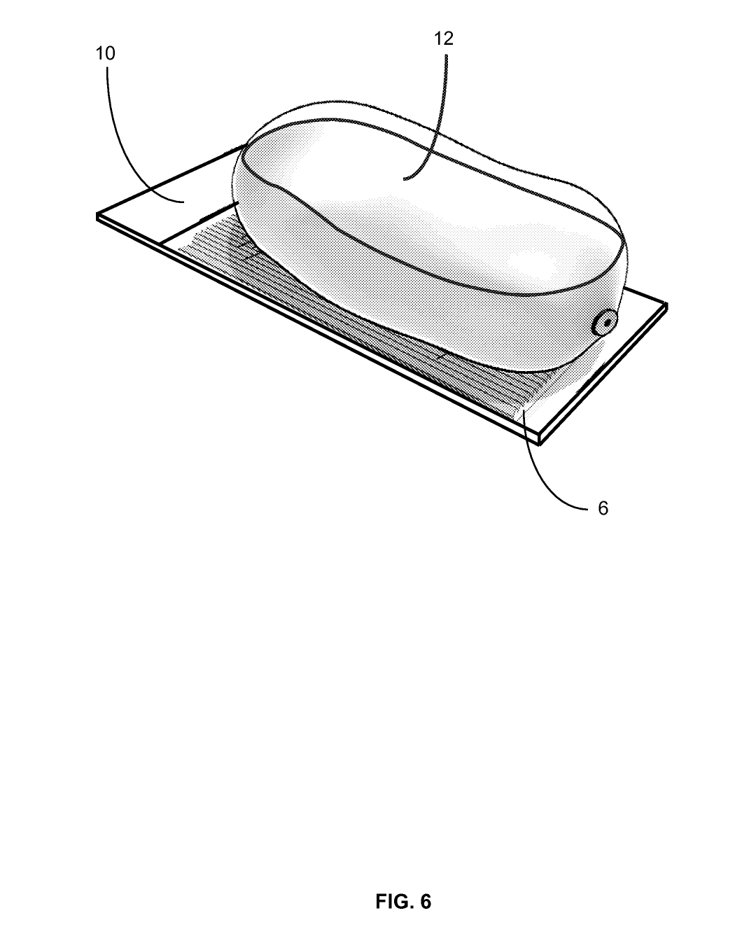

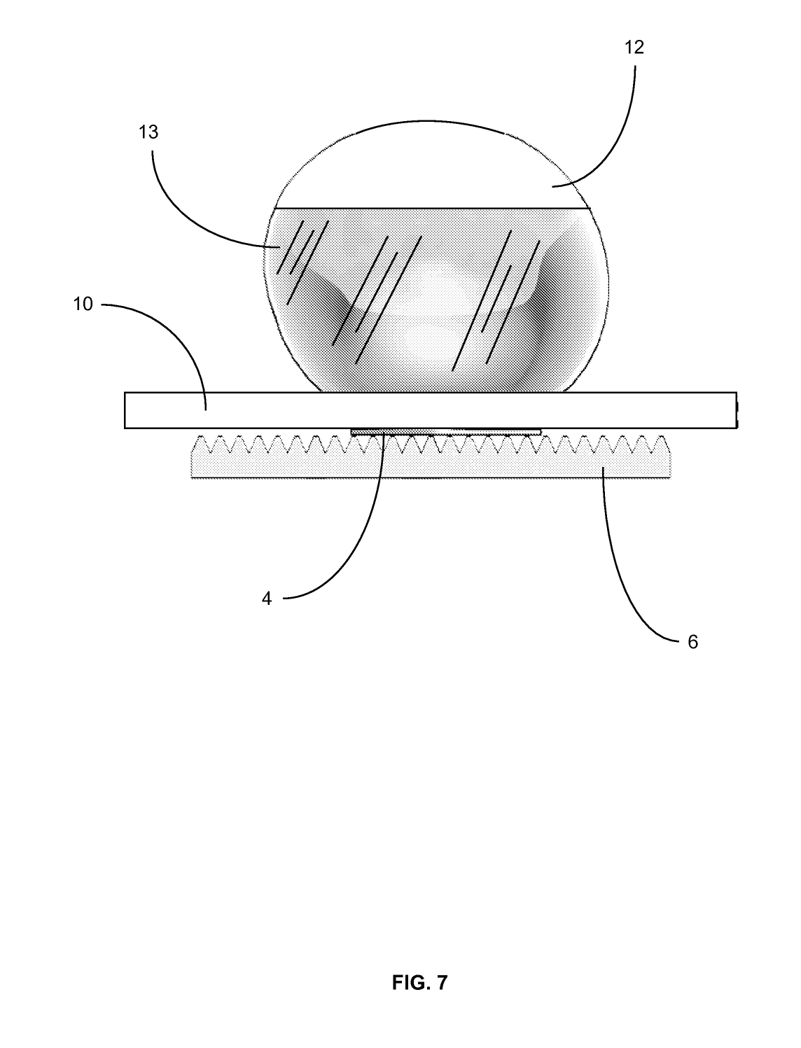

In order to transfer the sections from the affixing block to the slide on top of it, a relatively warmed heating element (FIG. 6; 12) (for example, room temperature) is transiently placed on top of the slide (FIG. 6; 10) which itself is on top of the affixing block (FIG. 6; 6). The heating element should be of a material that does not rapidly equilibrate to the lower temperature of the cryostat and should evenly distribute the heat applied to the back of the slide. A room temperature liquid filled balloon (FIG. 7; 12) with enough liquid (FIG. 7; 13) to maintain heat is one such embodiment of the invention. The application of the balloon to the back side of the slide (FIG. 7; 10) will evenly distribute the heat on the slide and unfreeze the sections (FIG. 7; 4) on the surface of the affixing block (FIG. 7; 6). The time of balloon application to the slide is dependent upon the thickness of the sections, thicker sections require longer application. However, the time of application of the heating element for most tissue section thicknesses should be no more than a few seconds. The ideal time duration of heating element application is the minimum required to induce adherence to the slide. However, longer then necessary applications of the heating element generally do not affect net adherence of the sections to the slide, but do effect the increase the subsequent time needed waiting for adherence to occur.

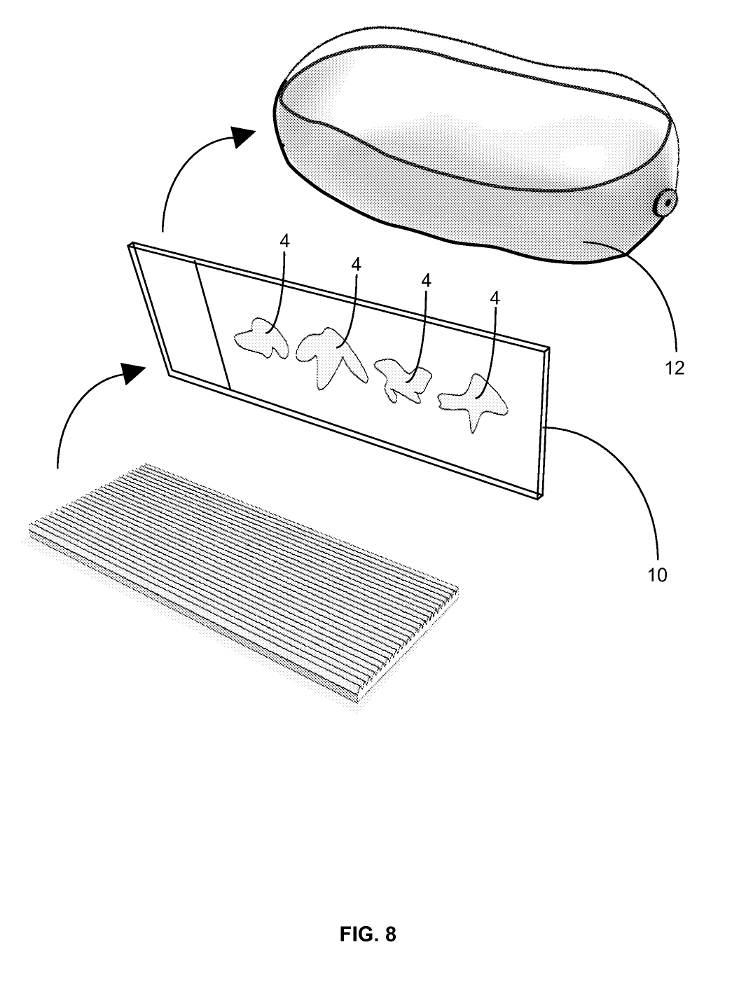

After the subsequent removal (FIG. 8; top arrow) of the balloon (FIG. 8; 12) , it will cause the sections (FIG. 8; 4) to attach to the slide (FIG. 8; 10) after a period of time. The time period required for adherence to the slide varies from seconds to minutes and is dependent on the duration of the application of the heating element. During this waiting period, there is a visible indication of when adherence of the sections to the slide occurs, in that after application of the heating element, generally the tissue sections change transparency, becoming more transparent. Upon adherence to the slide, the tissue sections at different times individually return to being more opaque.

After adherence, the slide and attached sections can be removed (FIG. 8; bottom arrow) from the affixing surface (FIG. 8; 6) , by removing the slide. Twisting of the slide relative to the affixing block, is particularly effective at ensuring full transference of the tissue sections from the affixing block to slides. Subsequent molecular biology procedures can then be performed on these slides.

We claim:

1. An apparatus for affixing frozen tissue sections onto slides comprising one or more teasing elements, an affixing block with one or more affixing surfaces, and a heating element.

2. The apparatus of claim 1 wherein said teasing element is a rod with a pointed end.

3. The apparatus of claim 1 wherein said teasing element is attached to a vacuuming device.

4. The apparatus of claim 1 wherein said heating element is a liquid filled balloon.

5. The apparatus of claim 1 wherein said heating element is controlled by a microprocessor.

6. The apparatus of claim 1 wherein said heating element is a solid block.

7. The apparatus of claim 1 wherein said affixing block and heating element are attached by a hinge.

8. The apparatus of claim 1 wherein said affixing block and heating element contain magnets.

9. The apparatus of claim 1 wherein said affixing surface is composed of a hydrophobic substance.

10. The apparatus of claim 1 wherein said affixing surface consists of indentations or bumps.

11. The apparatus of claim 10 wherein said indentations or bumps have a width of 25 micrometers to 5 millimeters.

12. The apparatus of claim 10 wherein said indentations or bumps are separated by a distance of 25 micrometers to 5 millimeters.

13. The apparatus of claim 10 wherein said indentations have a depth of 25 micrometers to 5 millimeters.

14. The apparatus of claims 10 wherein said indentations are grooves or holes.

15. The apparatus of claim 10 wherein said bumps have a height of 25 micrometers to 5 millimeters.

16. A method of affixing frozen tissue sections to a slide comprising the steps of:
placing said slide, said teasing implement and said affixing block inside the cryostat and allowing them to equilibrate to the temperature of the cryostat.
cutting frozen slices of said tissue sections and embedding medium.
separating said tissue sections from said medium using said teasing implement.
transferring said tissue sections with said teasing implement to said affixing block on top of said fixing surface.
placing said slide on top of said affixing surface and said sections.
placing said heating element on top of said slide for a duration of time.
removing said heating element.
waiting for said sections to adhere to said slide for a duration of time.
removing said slide with said sections from said affixing block.
performing subsequent molecular biology procedures with said slide.

17. The method of claim 16 in which the duration of time for placing the affixing said heating element on top of said slide consists from 1-100 seconds.

18. The method of claim 16 in which the duration of time for waiting for said sections to adhere to said slide consists from 1-1000 seconds.
